SEQUENCE LISTING (12 sequences)

SEQ ID NO: 1 - DNA, 65 bp, Artificial Sequence
  Nucleotide sequence of the signal peptide SEQ ID NO 1 (Ompc)
  misc_feature: SEQ ID NO 1 (nucleotide sequence of the signal peptide SEQ ID NO 1 Ompc)
atgaaagtgaaggttctgagcctgttggtccctgccctgttagttgccggtgctgcaaacgcagg

SEQ ID NO: 2 - protein, 21 aa, Artificial sequence
  MISC_FEATURE (1)..(21): SEQ ID NO 2 Aminoacid sequence of the signal peptide OmpC
MKVKVLSLLVPALLVAGAANA

SEQ ID NO: 3 - DNA, 1674 bp, Artificial sequence
  SEQ ID NO 3 Nucleotide sequence of the protein CRM197 comprising the nucleotide sequence of the signal peptide SEQ ID NO 1 (Ompc)
  CDS (1)..(1674): SEQ ID NO 3 Nucleotide sequence of the protein CRM197 comprising the nucleotide sequence of the signal peptide SEQ ID NO 1 (Ompc)
  sig_peptide (1)..(63): Nucleotide sequence of the signal peptide SEQ ID NO 1 (Ompc)
  misc_feature (64)..(1674): Nucleotide sequence for the mature protein CRM197
atgaaagtgaaggttctgagcctgttggtccctgccctgttagttgccggtgctgcaaacgcaggcgctgacgatgttgtggatagcagcaaatctttcgttatggaaaacttctctagctatcacggtacgaaaccgggttacgtggatagcattcaaaagggtattcagaagccgaagtccggcacccagggtaactatgatgacgactggaaagaattctatagcaccgacaacaaatacgacgcagccggctacagcgtggacaacgaaaatccgctgagcggtaaggccggtggcgttgttaaagtcacgtacccgggtctgaccaaagtgctggcgttgaaagtcgataacgctgaaacgattaaaaaagaactgggtctgagcctgaccgagccgctgatggaacaggtcggtactgaagaattcattaaacgctttggtgacggtgcatctcgcgtggttctgagcctgccgttcgccgagggctctagcagcgttgagtacatcaacaactgggaacaggcgaaggccttgtccgttgagctggaaatcaactttgaaacccgtggcaagcgcggtcaagacgcgatgtatgagtacatggcgcaagcgtgtgcgggcaatcgtgtacgtcgtagcgttggcagcagcctgtcttgcattaatctggactgggacgtgattagagataagaccaagacgaagattgagagcctgaaagaacatggcccaatcaagaataagatgagcgagagcccgaataaaacggtcagcgaagagaaagcgaaacagtacctggaagaattccaccagaccgcactggagcatcctgagctgagcgagctgaaaaccgtgactggcacgaatcctgttttcgctggtgccaactacgccgcatgggcagttaacgttgcgcaagtgatcgattccgaaacggctgataatctggagaaaaccaccgcggccctgtcgatcttgccgggtattggcagcgtgatgggtatcgcggatggcgcagtccatcacaataccgaagaaatcgttgcgcagtctattgcattgagctcgttaatggtcgcgcaagcgattccgcttgtcggcgaactggttgatatcggttttgcagcgtacaacttcgtcgagagcattatcaacttgttccaagttgttcacaatagctacaaccgtccggcctatagcccgggtcacaagacccagccattcttgcacgacggttatgctgtgagctggaacactgtcgaggatagcatcattcgtaccggtttccagggtgagagcggtcacgacatcaaaattactgcggagaatacgccgctgccgatcgcgggtgtgctgctgccgaccatcccgggtaaactggatgtcaataagtccaagacgcatatttccgtgaatggtcgcaaaatccgcatgcgctgtcgtgcgattgacggcgatgtgaccttttgccgtccgaaaagcccggtctacgtcggcaacggtgtgcacgcaaatctccatgttgcgtttcatcgttcgagctccgaaaagatccacagcaatgaaatctccagcgatagcattggtgttctgggctatcagaaaacggtcgaccacaccaaggttaatagcaagctgtcactgttttttgagatcaagagctaatga

SEQ ID NO: 4 - protein, 556 aa, Artificial sequence
  Synthetic construct SEQ ID NO 4 aminoacid sequence of the protein CRM197 comprising the aminoacid sequence of the signal peptide SEQ ID NO 1 (Ompc)
MKVKVLSLLVPALLVAGAANAGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 5 - DNA, 42 bp, Artificial sequence
  IPTG-inducible promoter sequence (SEQ ID NO 5)
  Promotor (1)..(42): IPTG-inducible promoter sequence (SEQ ID NO 5)
taatacgactcactataggggaattgtgagcggataacaatt

SEQ ID NO: 6 - DNA, 20 bp, Artificial sequence
  SEQ ID NO 6 Consensus sequence for the Ribosome Binding Site of the expression vector (medium- strength Ribosome Binding Site, average RBS)
  RBS (1)..(20): SEQ ID NO 6 Consensus sequence for the Ribosome Binding Site of the expression vector (medium- strength Ribosome Binding Site, average RBS)
aattaggtaaaaaataaaaa

SEQ ID NO: 7 - DNA, 114 bp, Artificial sequence
  Rhamnose-inducible promoter sequence (SEQ ID NO 7)
  promotor (1)..(114): Rhamnose-inducible promoter sequence (SEQ ID NO 7)
caccacaattcagcaaattgtgaacatcatcacgttcatctttccctggttgccaatggcccattttcctgtcagtaacgagaaggtcgcgaattcaggcgctttttagactgg

SEQ ID NO: 8 - DNA, 63 bp, Artificial sequence
  Nucleotide sequence of the signal peptide OmpA (SEQ ID NO 8)
  misc_feature (1)..(63): Nucleotide sequence of the signal peptide OmpA (SEQ ID NO 8)
atgaaaaagacagctatcgcgattgcagtggcactggctggtttcgctaccgtagcgcaggca

SEQ ID NO: 9 - protein, 21 aa, Artificial sequence
  Aminoacid sequence of the signal peptide OmpA (SEQ ID NO 9)
  SIGNAL (1)..(21): Aminoacid sequence of the signal peptide OmpA (SEQ ID NO 9)
MKKTAIAIAVALAGFATVAQA

SEQ ID NO: 10 - DNA, 5669 bp, Artificial sequence
  SEQ ID NO 10 Complete sequence of the construct BL21(DE3)/pD431-MR-OmpC-CRM197 also labelled BL21(DE3) / pLM7-OC-CRM). SEQ ID NO 10 harbors a low-copy number replication origin, Ori_p15a, from position 2441 to position 3267 of SEQ ID NO 10.
  misc_feature (1)..(5669): SEQ ID NO 10 omplete sequence of the construct BL21(DE3)/pD431-MR-OmpC-CRM197 also labelled BL21(DE3) / pLM7-OC-CRM)
ggttgaggtctcaccccctagcataaccccttggggcctctaaacgggtcttgaggggttttttgcccctgagacgcgtcaatcgagttcgtacctaagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcggcgcgccatcgaatggcgcaaaacctttcgcggtatggcatgatagcgcccggaagagagtcaattcagggtggtgaatatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtcccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttccgcgaaattaatacgactcactataggggaattgtgagcggataacaattcccctctagaaataattttgtttaacttttagagaccaaattaggtaaaaaataaaaaatgaaagtgaaggttctgagcctgttggtccctgccctgttagttgccggtgctgcaaacgcaggcgctgacgatgttgtggatagcagcaaatctttcgttatggaaaacttctctagctatcacggtacgaaaccgggttacgtggatagcattcaaaagggtattcagaagccgaagtccggcacccagggtaactatgatgacgactggaaagaattctatagcaccgacaacaaatacgacgcagccggctacagcgtggacaacgaaaatccgctgagcggtaaggccggtggcgttgttaaagtcacgtacccgggtctgaccaaagtgctggcgttgaaagtcgataacgctgaaacgattaaaaaagaactgggtctgagcctgaccgagccgctgatggaacaggtcggtactgaagaattcattaaacgctttggtgacggtgcatctcgcgtggttctgagcctgccgttcgccgagggctctagcagcgttgagtacatcaacaactgggaacaggcgaaggccttgtccgttgagctggaaatcaactttgaaacccgtggcaagcgcggtcaagacgcgatgtatgagtacatggcgcaagcgtgtgcgggcaatcgtgtacgtcgtagcgttggcagcagcctgtcttgcattaatctggactgggacgtgattagagataagaccaagacgaagattgagagcctgaaagaacatggcccaatcaagaataagatgagcgagagcccgaataaaacggtcagcgaagagaaagcgaaacagtacctggaagaattccaccagaccgcactggagcatcctgagctgagcgagctgaaaaccgtgactggcacgaatcctgttttcgctggtgccaactacgccgcatgggcagttaacgttgcgcaagtgatcgattccgaaacggctgataatctggagaaaaccaccgcggccctgtcgatcttgccgggtattggcagcgtgatgggtatcgcggatggcgcagtccatcacaataccgaagaaatcgttgcgcagtctattgcattgagctcgttaatggtcgcgcaagcgattccgcttgtcggcgaactggttgatatcggttttgcagcgtacaacttcgtcgagagcattatcaacttgttccaagttgttcacaatagctacaaccgtccggcctatagcccgggtcacaagacccagccattcttgcacgacggttatgctgtgagctggaacactgtcgaggatagcatcattcgtaccggtttccagggtgagagcggtcacgacatcaaaattactgcggagaatacgccgctgccgatcgcgggtgtgctgctgccgaccatcccgggtaaactggatgtcaataagtccaagacgcatatttccgtgaatggtcgcaaaatccgcatgcgctgtcgtgcgattgacggcgatgtgaccttttgccgtccgaaaagcccggtctacgtcggcaacggtgtgcacgcaaatctccatgttgcgtttcatcgttcgagctccgaaaagatccacagcaatgaaatctccagcgatagcattggtgttctgggctatcagaaaacggtcgaccacaccaaggttaatagcaagctgtcactgttttttgagatcaagagctaatga

SEQ ID NO: 11 - DNA, 5631 bp, Artificial Sequence
  SEQ ID NO 11 Complete sequence of the construct BL21(DE3)/pD451-SR-OmpC-CRM197 (also labeled BL21(DE3)/pHS7-OC-CRM. SEQ ID NO 11 harbors a high-copy number replication origin, Ori_pUC, from position 2500 to position of SEQ ID NO 11.
  misc_feature (1)..(5631): SEQ ID NO 11 Complete sequence of the construct BL21(DE3)/pD451-SR-OmpC-CRM197 (also labeled BL21(DE3)/pHS7-OC-CRM
ggttgaggtctcaccccctagcataaccccttggggcctctaaacgggtcttgaggggttttttgcccctgagacgcgtcaatcgagttcgtacctaagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcggcgcgccatcgaatggcgcaaaacctttcgcggtatggcatgatagcgcccggaagagagtcaattcagggtggtgaatatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtgactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttccgcgaaattaatacgactcactataggggaattgtgagcggataacaattcccctctagaaataattttgtttaactttttgagaccttaaggaggtaaaaaatgaaagtgaaggttctgagcctgttggtccctgccctgttagttgccggtgctgcaaacgcaggcgctgacgatgttgtggatagcagcaaatctttcgttatggaaaacttctctagctatcacggtacgaaaccgggttacgtggatagcattcaaaagggtattcagaagccgaagtccggcacccagggtaactatgatgacgactggaaagaattctatagcaccgacaacaaatacgacgcagccggctacagcgtggacaacgaaaatccgctgagcggtaaggccggtggcgttgttaaagtcacgtacccgggtctgaccaaagtgctggcgttgaaagtcgataacgctgaaacgattaaaaaagaactgggtctgagcctgaccgagccgctgatggaacaggtcggtactgaagaattcattaaacgctttggtgacggtgcatctcgcgtggttctgagcctgccgttcgccgagggctctagcagcgttgagtacatcaacaactgggaacaggcgaaggccttgtccgttgagctggaaatcaactttgaaacccgtggcaagcgcggtcaagacgcgatgtatgagtacatggcgcaagcgtgtgcgggcaatcgtgtacgtcgtagcgttggcagcagcctgtcttgcattaatctggactgggacgtgattagagataagaccaagacgaagattgagagcctgaaagaacatggcccaatcaagaataagatgagcgagagcccgaataaaacggtcagcgaagagaaagcgaaacagtacctggaagaattccaccagaccgcactggagcatcctgagctgagcgagctgaaaaccgtgactggcacgaatcctgttttcgctggtgccaactacgccgcatgggcagttaacgttgcgcaagtgatcgattccgaaacggctgataatctggagaaaaccaccgcggccctgtcgatcttgccgggtattggcagcgtgatgggtatcgcggatggcgcagtccatcacaataccgaagaaatcgttgcgcagtctattgcattgagctcgttaatggtcgcgcaagcgattccgcttgtcggcgaactggttgatatcggttttgcagcgtacaacttcgtcgagagcattatcaacttgttccaagttgttcacaatagctacaaccgtccggcctatagcccgggtcacaagacccagccattcttgcacgacggttatgctgtgagctggaacactgtcgaggatagcatcattcgtaccggtttccagggtgagagcggtcacgacatcaaaattactgcggagaatacgccgctgccgatcgcgggtgtgctgctgccgaccatcccgggtaaactggatgtcaataagtccaagacgcatatttccgtgaatggtcgcaaaatccgcatgcgctgtcgtgcgattgacggcgatgtgaccttttgccgtccgaaaagcccggtctacgtcggcaacggtgtgcacgcaaatctccatgttgcgtttcatcgttcgagctccgaaaagatccacagcaatgaaatctccagcgatagcattggtgttctgggctatcagaaaacggtcgaccacaccaaggttaatagcaagctgtcactgttttttgagatcaagagctaatga

SEQ ID NO: 12 - protein, 535 aa, Artificial sequence
  MISC_FEATURE (1)..(535): SEQ ID NO 12 Aminoacid sequence of the mature protein CRM197 (without the signal peptide SEQ ID NO 2)
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

(19) United States
(12) Patent Application Publication
(10) Pub. No.: US 2025/0326804 A1
(43) Pub. Date: Oct. 23, 2025

(54) METHOD FOR PRODUCING A PERIPLASMIC FORM OF THE PROTEIN CRM197

(71) Applicants: XPRESS BIOLOGICS, Herstal (BE); CURAVAC EUROPE, Rixensart (BE)

(72) Inventors: Marc DAUKANDT, Bilstain (BE); Nicolas HAVELANGE, Bruxelles (BE); Stéphane HUBERTY, Rixensart (BE); Philippe LEDENT, Liège (BE); Christian RODRIQUEZ, Spa (BE)

(21) Appl. No.: 18/278,778

(22) PCT Filed: Feb. 28, 2022
(86) PCT No.: PCT/EP2022/054914

§ 371 Date: Aug. 24, 2023

(30) Foreign Application Priority Data
Feb. 26, 2021 (BE) BE2021/5137

(51) Int. Cl. C07K 14/34; C12N 1/20; C12N 15/62; C12N 15/70; C12P 19/34; C12R 1/19

(52) U.S. Cl. CPC C07K 14/34; C12N 1/20; C12N 15/625; C12N 15/70; C12P 19/34; C07K 2319/02; C12R 2001/19

(57) ABSTRACT

The present invention relates to a method for producing a periplasmic form of SEQ ID NO: 12, to the expression vector encoding SEQ ID NO: 12 and signal sequence SEQ ID NO: 2, enabling the targeting of SEQ ID NO: 12 towards the periplasmic space, as well as to the strain transformed by the expression vector.

### CROSS-REFERENCE TO RELATED APPLICATIONS

The present application is a National Stage Filing of PCT International Application No. PCT/EP2022/054914 filed on Feb. 28, 2022, which claims priority to Belgian application No. BE2021/5137, filed with the Belgian Patent Office on Feb. 26, 2021, which applications are incorporated herein by reference in their entirety.

### SEQUENCE STATEMENT

This application contains a Sequence Listing, which was submitted electronically in ASCII format and is hereby incorporated by reference in its entirety. Said ASCII copy, created and filed in PCT/EP2022/054914 has the name “2023-08-file 24_186780015FPWO_SEQ_LISTING.txt” and a file size 37,798 bytes (36.9 KB) and was modified at Line <110> to include the filing date of this application.

### FIELD OF THE INVENTION

The present invention relates to a method for producing a periplasmic form of the protein CRM197, to the expression vector encoding protein CRM197 and signal sequence, OmpC, enabling the targeting of protein CRM197 towards the periplasmic space, as well as to the strain transformed by the expression vector.

### SUMMARY OF THE INVENTION

The recombinant protein CRM197, Cross Reacting Material, or CRM, may be used, for example, as a carrier protein in conjugate vaccines. A conjugate vaccine is a type of vaccine combining a slightly immunogenic antigen with a “carrier” protein that has T epitopes recognised by the immune system, in order to obtain a stronger response to the antigen.

CRM197 is a non-toxic mutant protein of diphtheria toxin. It is used as a carrier protein for haptens such as polysaccharides to enable an immunogenic response. A hapten is one of the components of an antigen which, when combined with a protein carrier, gives it sufficient antigenicity to generate a humoral immune response.

The protein CRM197 is a detoxified form of diphtheria toxin where a substitution of the glycine at position 52 with a glutamic acid abolishes the ADP-ribosyltransferase activity of the native toxin and renders it non-toxic. The protein CRM197 comprises a single polypeptide chain of 535 amino acids, with a molecular weight of 58.4 kDa.

The CRM197 gene was initially cloned in Corynebacterium diphtheriae, the pathogenic bacterium that causes diphtheria and contains the native toxin. As the native toxin, CRM197 is only very slightly secreted. As a result, significant efforts have been made to produce the protein CRM197 in other bacteria.

Today, the protein CRM197 is used as a carrier protein in several conjugate vaccines. For example, Hibtiter™, a vaccine for protection against Haemophius influenzae type b, approved by the FDA (Food and Drug Administration) in 1990, was the first conjugate vaccine to use the protein CRM197 as a carrier protein; the hapten is a sugar.

The protein CRM197 also has a binding site for HB-EGF (heparin-binding epidermal growth factor-like growth factor), a member of the EGF family. As this EGF receptor is over-expressed in numerous cancer cells, literature indicates that the protein CRM197 could also be used as a potential agent in targeted anti-cancer therapies.

Demand for industrial production of the protein CRM197 is therefore increasing.

Unfortunately, when it comes to producing CRM197, it is still difficult to achieve high and stable expression levels of the protein CRM197 in the native wild-type strain of E. coli BL21 bacteria. An insoluble form of the protein CRM197 can be produced at a relatively high level by fermentation in E. coli, but only a very small fraction of this insoluble form of the protein can be converted into a soluble form.

### PRIOR ART

Production of the protein CRM197 in a K12 strain of E. coli is known from document EP3113800. The document relates to a method for producing a recombinant protein CRM197 in an E. coli host cell. In several embodiments, the method comprises incubating a strain of E. coli having a reduced genome comprising an expression vector comprising a nucleic acid sequence encoding the protein CRM197 operably linked to an expression control sequence under conditions suitable for expression of the recombinant protein CRM197. A significant increase in CRM197 yield is obtained in an E. coli host cell with a reduced genome according to EP3113800 compared with production in wild-type strains of E. coli such as BL21.

Document EP3170837 provides an improved method for producing a bacterial toxin by periplasmic expression comprising the steps of a) growing a culture of the bacterial host cell containing an expression vector wherein particular signal peptide sequences are linked to the sequence of a bacterial toxin and b) inducing expression of the polypeptide containing particular signal peptide sequences linked to a bacterial toxin such that a bacterial toxin is expressed and secreted in the periplasm.

Document EP2445930 provides a method for producing the protein CRM197 and similar proteins, as an alternative to using the micro-organism Corynebacterium diphtheriae. According to the procedure described in this document, the protein of interest may be obtained in large quantities both for basic research and for medico-therapeutic applications.

Document EP3444269 describes an expression system for producing a diphtheria toxin polypeptide in an E. coli strain deficient in rhamnose catabolismhe document describes an expression system comprising a rhamnose-inducible promoter sequence and an expression system comprising a first portion with a signal sequence and a second portion comprising the diphtheria toxin sequence.

Meanwhile, document WO2011/126811A2 describes a method for producing a recombinant protein in Pseudomonas.

Philippe Goffin et al., Biotechnol. J. 2017, 12, 1700168, DOI 10.1002/biot.201700168, have also shown that decoupling the growth of the micro-organism and the production of the protein CRM197 may promote periplasmic production of the protein CRM197.

### BRIEF DESCRIPTION OF THE DRAWINGS

Some exemplary embodiments of the invention will now be described in more detail with the aid of the drawings, in which:

FIG. 1 shows the various steps of producing the protein SEQ ID NO: 12 by fermentation;

FIG. 2 shows the various steps of purifying the protein SEQ ID NO: 12; and

FIG. 3 shows an expression of the protein SEQ ID NO: 12.

### DETAILED DESCRIPTION OF THE INVENTION

Unfortunately, although some documents extol the virtues of their technology, there is currently no method on the market for producing CRM197 that is easily exploitable and industrially profitable, regardless of the scale of industrial production.

There is therefore a need for an alternative vector and an alternative strain transformed by this vector that enable a high and stable expression level of the soluble form of the protein CRM197 and a simple method for purifying the protein CRM197 so that it can be used industrially and is accessible to the entire market.

Given the growing global demand for the protein CRM197, one recurring problem with producing this protein is obtaining a high production yield that is stable over time and is as simple as possible, in order to achieve production costs that are accessible to the entire market.

To reduce these disadvantages and difficulties, the present invention provides an expression system that enables a high expression yield that is stable over time and that has optimised ratios between the rate of synthesis and that of secretion of the protein CRM197 (SEQ ID NO: 12) as well as a purification yield that enables SEQ ID NO: 12 to be commercially used and made available as a protein/peptide for subsequent development of vaccines or targeted therapies.

Firstly, the inventors sought to produce SEQ ID NO: 12 in yeast, which is advantageous for downstream purification steps; unfortunately, this did not work.

Likewise, the inventors used plasmids with a very high copy level or with an optimal promoter, so as to enable massive synthesis of SEQ ID NO: 12 in E. coli or in the periplasmic space thereof. This did not work.

After the various preliminary steps, the inventors concluded that an expression system was needed in the periplasmic space of E. coli, but with a well-calibrated level, high enough to be used on a large scale, but not too high to avoid aggregation in E. coli.

To do this, having identified the numerous problems above, the inventors actively sought the best culture and production conditions, including the best genetic constructs (transcription control, translation control, periplasmic targeting sequence).

More specifically, the present invention provides an expression vector in a Gram-negative prokaryote with the following features:
(i) a low copy number origin of replication, the low copy number being defined by a plasmid copy number of 20 per cell or less,
(ii) a promoter sequence inducible with an exogenous inducing agent,
(iii) a medium strength ribosome binding site, the medium strength ribosome binding site corresponds to an mRNA sequence modified to have a reduced ribosome binding,
(iv) a sequence encoding a protein having at least 95% identity to the full-length SEQ ID NO: 12,
(v) a sequence encoding a signal peptide having a sequence identity to SEQ ID NO: 2 of at least 95%, the signal peptide being arranged to enable a targeting of SEQ ID NO: 12 towards a periplasmic space.

Advantageously, the glutamic acid residue at position 52 of SEQ ID NO: 12 is retained. A mutation replacing the native glycine of SEQ ID NO: 12 at position 52 with a glutamic acid provides a detoxified SEQ ID NO: 12 peptide.

Particularly advantageously, the inducible promoter sequence has at least 95% identity to SEQ ID NO: 5 over the entire length and is specifically recognised by a T7 phage RNA polymerase.

Favourably, the medium strength ribosome binding site is defined by a consensus sequence SEQ ID NO: 6 or by the consensus sequence SEQ ID NO: 6 comprising an addition and/or a deletion and/or a point mutation of 1 or 2 or 3 or 4 or 5 nucleotides, the SEQ ID NO: 6 being located upstream of a translation initiation site.

The present invention also relates to a transformed strain of E. coli bacteria comprising the expression vector. The E. coli BL21 T7XB strain has been filed on behalf of Xpress Biologicals at the BCCM, ‘Belgian Co-ordinated Collections of Micro-organisms’ in Ghent on 23/12/2020 under the reference LMBP 12507.

The transformed strain of E. coli BL21 T7XB bacteria with the expression vector which comprises a low copy number origin of replication, defined by a plasmid copy number between 1 and 20 per cell, and a medium strength ribosome binding site, i.e. having an mRNA sequence modified so that ribosome binding is reduced, makes it possible to obtain a very good ratio between the expression level and secretion of SEQ ID NO: 12 and ensures a high production yield while being stable over time.

Preferably, a strain of E. coli BL21 bacteria defined by a deletion of the ADE3 prophage with the exception of the genes required for expressing T7 phage RNA polymerase: E. coli BL21 (ADE3) or E. coli BL21 T7XB. The advantages of this deletion are that there is no risk of reactivating the prophage, it offers great flexibility for production without the risk of cross-contamination and it provides high plasmid stability.

The present invention also relates to a method for producing in the periplasmic space a peptide having at least 95% identity to SEQ ID NO: 12 over the entire length thereof comprising:
(i) transforming a strain of E. coli by a vector enabling expression of SEQ ID NO: 12 in the periplasmic space of the transformed E. coli strain, the expression being inducible by an exogenous agent,
(ii) culturing the transformed E. coli strain at a substantially constant temperature, for example 37° C.±1° C., and a constant pH under conditions where SEQ ID NO: 12 is not synthesised by the transformed E. coli strain,
(iii) an induction phase for a predetermined time period in the presence of the exogenous agent and at a reduced temperature to synthesise and secrete SEQ ID NO: 12 in the periplasmic space in a slow and controlled manner, advantageously in order to reduce toxicity, metabolic overload and/or saturation of secretion pathways,
(iv) optionally harvesting the culture after the induction phase and after cooling, for example, to 15° C.±0.5° C.,
(v) optionally collecting the cultured bacteria containing SEQ ID NO: 12 in the periplasmic space,
(vi) optionally performing periplasmic extraction of SEQ ID NO: 12 including separating a solid pellet containing the bacteria and a supernatant containing the components of the periplasmic space and SEQ ID NO: 12, and
(vii) optionally purifying the protein SEQ ID NO: 12.

The present invention therefore provides a method for producing SEQ ID NO: 12 that is optimised for stable and sufficient expression where the temperature and pH remain substantially constant for the entire induction phase, which simplifies the production method, minimises the risk of errors and also ensures optimal yield in terms of production costs. The substantially constant temperature is a temperature compatible with an optimal growth metabolism of the transformed E. coli strain.

Preferably, the E. coli strain is the BL21 strain defined by a deletion of the ADE3 prophage with the exception of the genes required for expressing T7 phage RNA polymerase.

Advantageously, production in the periplasmic space of a peptide having at least 95% identity to SEQ ID NO: 12 over the entire length thereof is provided by a targeting signal peptide in the periplasmic space, the signal peptide having a sequence identity to SEQ ID NO: 2 of at least 95%.

Preferably, the reduced temperature is 23° C.±0.5° C. and/or the predetermined time period of the induction phase is between 15 and 25 hours, preferably between 18 and 22 hours and more preferably around 20 hours and/or the pH of the induction phase is between 6.8 and 7.5, preferably between 7.0 and 7.2.

Preferably, expression inducible by an exogenous agent is performed by an on/off induction system and the exogenous agent is isopropyl-β-D-1-thiogalactopyranoside. Isopropyl-β-D-1-thiogalactopyranoside is also known as IPTG.

Advantageously, the culture and induction phase are free of antibiotics such as kanamycin, ampicillin, streptomycin, chloramphenicol or tetracycline.

Preferably, the expression vector is of a low copy number defined by a plasmid copy number of 20 per cell or less and/or the ribosome binding sequence is of medium strength.

Advantageously, purification of the protein SEQ ID NO: 12 comprises the successive steps of:
(i) clarifying the supernatant from the periplasmic extraction containing the components of the periplasmic space and the protein SEQ ID NO: 12 by at least one filtration step, preferably two filtration steps and recovering a filtrate comprising SEQ ID NO: 12, optionally,
(ii) a series of chromatographic steps arranged to recover SEQ ID NO: 12 in a fraction to be retained and the contaminants in one or more fractions to be removed,
(iii) optionally, removing residual endotoxins, and
(iv) optionally, sterilising the endotoxin-free fraction comprising SEQ ID NO: 12 to be retained.

Advantageously, periplasmic extraction is periplasmic extraction by osmotic shock.

Preferably, periplasmic extraction comprises the steps of:
(i) suspending the pellet formed by the collected bacteria in a hypertonic solution to form a hypertonic suspension of bacteria,
(ii) transferring the hypertonic suspension of bacteria into a second mixing container through a diffusion ring and by means of a transfer tube and a first pump,
(iii) feeding said mixing container with a hypotonic solution by means of a transfer tube and a second pump, so as to cause an osmotic shock to the bacteria in the hypertonic suspension in the second mixing container, the osmotic shock releasing the contents of the periplasmic space into the solution of the second mixing container,
(iv) draining the mixture so that the residence time of the bacteria in the suspension of bacteria in the second container is between 10 seconds and one minute, more preferably around 30 seconds, and so that the volume of the mixture in the second container is kept constant, the draining being carried out by a tube and a third peristaltic pump.

Preferably, the series of chromatographic steps comprises a first chromatography on an anion exchange resin and a second chromatography on a hydrophobic resin.

Lastly, the present invention relates to a method for selecting a signal peptide so as to optimise production of a protein of interest in the periplasmic space of E. coli bacteria, wherein:
a genetic construct is generated, comprising a rhamnose-inducible promoter having at least 95% sequence identity to SEQ ID NO: 7 controlling the expression of the protein of interest arranged with the signal peptide to be tested,
the E. coli bacteria are transformed with the genetic construct,
several parallel cultures of the transformed E. coli bacteria are produced in a rhamnose-free culture medium,
the transformed E. coli bacteria is cultured in the presence of a determined concentration of rhamnose so as to obtain a low induction of expression of the protein of interest and another culture of the transformed E. coli bacteria in the presence of another determined concentration of rhamnose so as to obtain a high induction of expression of the protein of interest and,
the abundance of the protein of interest in the periplasmic space is compared and, optionally, the deleterious effects to metabolism selected from the group consisting of plasmid loss, reduction in bacterial biomass, formation of cytosolic aggregates or degradation products and partial cleavage of the signal peptide are quantified and,
the signal peptide is selected according to the abundance of the protein of interest in the periplasmic space and, optionally, by rejecting signal peptides associated with excessively high deleterious effects.

The deleterious effects to metabolism are quantified, for example by establishing a score associated with the signal peptide and protein of interest pairing where several deleterious effects are taken into account such as plasmid loss, reduction in bacterial biomass, formation of cytosolic aggregates or degradation products and partial cleavage of the signal peptide. In a normal process of periplasmic expression of a protein of interest, the signal peptide associated with this protein of interest is cleaved during transport to the periplasmic space. Partial cleavage of the signal peptide may therefore cause an undesired change to the functionality of the protein of interest.

Other features, details and advantages of the invention will emerge from the description given below, which is non-limiting and refers to the accompanying drawings.

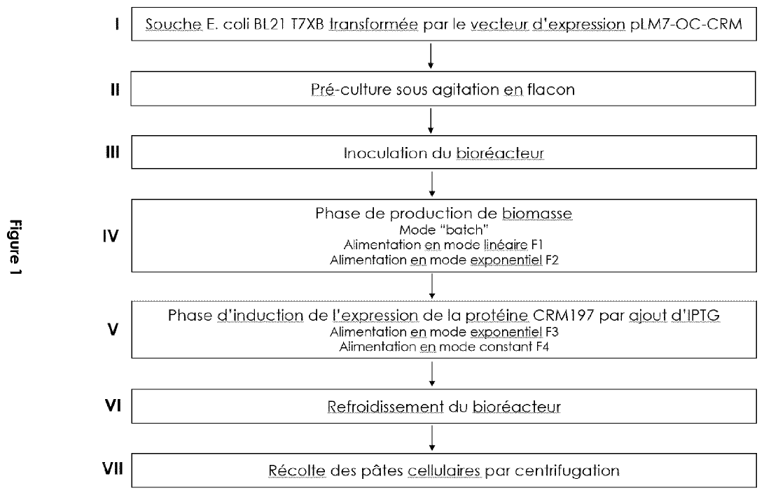

FIG. 1 shows the various steps of producing the protein SEQ ID NO: 12 by fermentation.

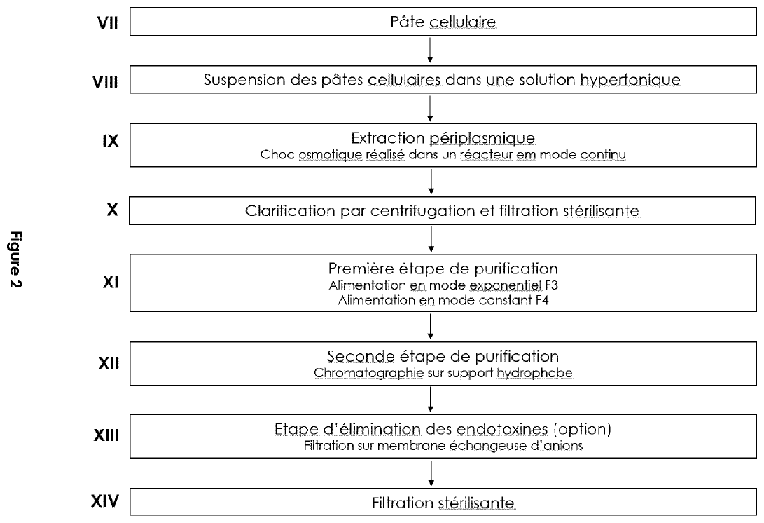

FIG. 2 shows the various steps of purifying the protein SEQ ID NO: 12.

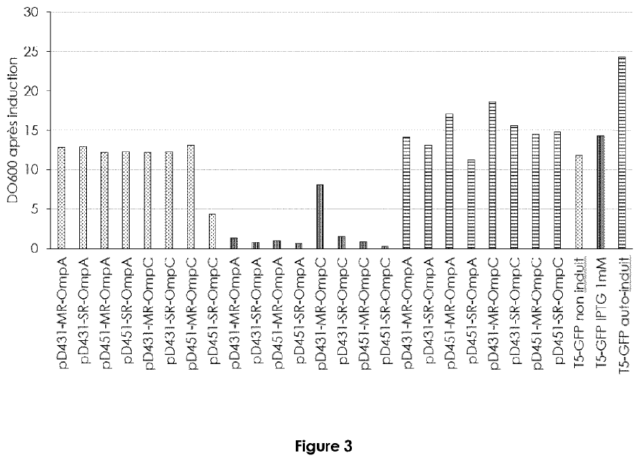

FIG. 3 shows an expression of the protein SEQ ID NO: 12 using an optical density measurement at 600 nm of E. coli BL21 (DE3) cultures inoculated with various expression vectors. Plasmid pD431: low plasmid copy number, plasmid pD451: high plasmid copy number. MR: medium ribosome binding strength, SR: high ribosome binding strength. Induction type: non-induced cultures (columns with black dots), cultures induced with IPTG (columns with vertical lines) or self-induced with lactose (columns with horizontal lines). The last 3 columns on the right: positive controls under the same induction conditions.

The E. coli BL21 T7XB strain transformed by expression vector pLM7-OC-CRM (step I in FIG. 1) was cultured at a constant temperature of 37° C.±0.5° C. and a constant pH of 7.0±0.2.

More particularly, the culture (steps II to IV in FIG. 1) of the E. coli BL21 strain transformed by the expression vector pLM7-OC-CRM at a constant temperature of 37° C.±0.5° C. then, on induction (step V in FIG. 1), at a constant temperature of 23±0.5° C. and a constant pH of 7.0±0.2 consists of 3 successive steps:
(i) preculture in a shake flask (step II in FIG. 1),
(ii) growth in ‘batch’ mode in a bioreactor (steps III and IV in FIG. 1),
(iii) growth in ‘fed-batch’ mode in the bioreactor (steps III and IV in FIG. 1).

The bioreactor is, for example, a 50 L bioreactor, fed via a 0.2 μm sterilising filter with 35 L of GYPSCI medium, glycerol and yeast extract.

Growth in “fed-batch” mode in the bioreactor occurs with a supply of oxygen such that a level of dissolved oxygen is greater than or equal to 30% saturation, and with constant stirring.

Both “batch” and “fed-batch” cultures are preferably carried out in the presence of an anti-foaming agent.

Once the induced expression of SEQ ID NO: 12, encoded by the vector pLM7-OC-CRM, and the secretion of SEQ ID NO: 12 in the periplasmic space are complete, the culture is then harvested after cooling to 15° C.±0.5° C. (steps VI and VII in FIG. 1). The culture is followed by collection of the bacteria containing SEQ ID NO: 12 in the periplasmic space by centrifugation (step VII in FIG. 1).

In relation to the purification of SEQ ID NO: 12, the cell pellet formed from the separated bacteria is then stored at low temperature, e.g. −20° C., or even lower (step VII in FIG. 2). The collected bacteria are then used as basic material for periplasmic extraction (steps VIII and IX in FIG. 2 and see Example 4). The clarified supernatant containing the components of the periplasmic space and SEQ ID NO: 12 is purified as described above (steps×to XIV in FIG. 2). More specifically, the series of chromatographic steps comprises a first chromatography on an anion exchange resin (step XI in FIG. 2) and a second chromatography on a hydrophobic resin (step XII in FIG. 2).

Advantageously, the filtration step that follows is an ultrafiltration step (step XIV in FIG. 2). The first step of the ultrafiltration method consists of concentrating SEQ ID NO: 12 to approximately 4.5 mg/mL and the second step consists of diafiltering the concentrated retentate with a 10-fold larger volume of formulation buffer (15 mM phosphate buffer, 5% sucrose, pH 7.35). The 30 kDa hollow fibre was selected because it corresponds to the fibre with the highest ‘cut-off’ value that can retain SEQ ID NO: 12.

If improving the purity of SEQ ID NO: 12 is desired, an additional step of removing residual endotoxins may be carried out (step XIII in FIG. 2), more particularly on a membrane containing quaternary ammonium groups in its coating.

### ILLUSTRATIVE EXAMPLES

### Example 1.—Preparation of the expression vector

To select the vector enabling periplasmic expression by E. coli, several signal peptides were fused at the N-terminal of SEQ ID NO: 12 within a rhamnose-inducible vector. After transforming E. coli BL21 (DE3) with these various combinations, the expression of SEQ ID NO: 12 in each clone was evaluated by immunoblot analysis for various rhamnose concentrations, and therefore for various induction levels.

Three criteria were taken into account to select combinations of SEQ ID NO: 12-signal peptide (CRM197-signal peptide) which will then be tested for expression after induction with IPTG: (1) intensity of the band corresponding to SEQ ID NO: 12 and having an apparent molecular weight of ˜58 kDa, (2) no or low proportion of SEQ ID NO: 12 still possessing the signal peptide (apparent molecular weight of ˜60 kDa) indicating saturation of the secretion pathway, (3) no or low presence of aggregation or degradation products (bands having apparent molecular weights greater than 97 kDa or between 14 and 58 kDa).

A selection matrix was created to select the combinations of signal peptide-SEQ ID NO: 12 which will then be tested for expression after induction with IPTG. Table 1 shows the allocation of scores as well as cumulative scores, calculated by multiplying the scores obtained for the three criteria and for the three induction levels. The highest cumulative score represents the construct expressing the largest amount of periplasmic SEQ ID NO: 12, in the absence of cytoplasmic SEQ ID NO: 12 and without degradation or aggregation.

Although the score of the construct TorT-SEQ ID NO: 12 is much higher than for the other variants, except at 11 mM, the cleavage of the signal peptide during secretion leaves an additional alanine residue at the beginning of the N-terminal of the protein SEQ ID NO: 12. The identity of the SEQ ID NO: 12 produced is not 100%, which led to the inventors discarding this variant. Two candidates were selected for screening in IPTG-inducible vectors: SEQ ID NO: 9-SEQ ID NO: 12 (OmpA-CRM197) and SEQ ID NO: 2-SEQ ID NO: 12 (OmpC-CRM197).

TABLE 1
Matrix used to select the combinations of
signal peptide - SEQ ID NO: 12 (CRM197)

| | Rhamnose concentration | | | | | | | | | |
|---|---|---|---|---|---|---|---|---|---|---|
| Signal | 0.55 mM | | | 5.5 mM | | | 11 mM | | | Cumulative |
| peptides | S1 | S2 | S3 | S1 | S2 | S3 | S1 | S2 | S3 | score |
| DsbA | 2 | 2 | 2 | 2 | 2 | 1 | 1 | 2 | 2 | 128 |
| EOX | 1 | 2 | 3 | 2 | 2 | 1 | 1 | 2 | 2 | 96 |
| LamB | 1 | 1 | 3 | 2 | 1 | 1 | 1 | 2 | 3 | 36 |
| MglB | 1 | 1 | 2 | 2 | 1 | 1 | 2 | 2 | 2 | 32 |
| MmAp | 2 | 1 | 2 | 3 | 1 | 1 | 2 | 2 | 2 | 96 |
| OmpA | 1 | 1 | 2 | 2 | 2 | 2 | 2 | 2 | 3 | 192 |
| OmpT | 1 | 1 | 3 | 2 | 1 | 2 | 2 | 1 | 1 | 24 |
| PelB | 1 | 2 | 3 | 2 | 1 | 2 | 2 | 1 | 1 | 48 |
| PhoA | 1 | 2 | 3 | 2 | 1 | 1 | 1 | 2 | 2 | 48 |
| SfmC | 3 | 2 | 1 | 1 | 2 | 1 | 2 | 2 | 1 | 48 |
| STII | 3 | 2 | 1 | 1 | 2 | 1 | 3 | 2 | 1 | 72 |
| TolB | 3 | 2 | 1 | 1 | 2 | 1 | 3 | 2 | 1 | 72 |
| TorT | 3 | 2 | 2 | 2 | 2 | 2 | 3 | 2 | 1 | 576 |
| OmpC | 3 | 1 | 2 | 2 | 1 | 1 | 3 | 2 | 2 | 144 |
| SipA | 3 | 2 | 1 | 2 | 1 | 1 | 2 | 2 | 1 | 48 |

Scores allocated (1-2-3) based on immunoblot results (Western Blots):
S1: score for criterion 1: intensity of the band corresponding to SEQ ID NO: 12.
S2: score for criterion 2: no/low proportion of protein still possessing the signal peptide.
S3: score for criterion 3: no/low presence of aggregation or degradation products.

Both selected combinations of peptide-SEQ ID NO: 12 were subcloned in a series of IPTG-inducible vectors where i) the origin of replication (copy number), ii) the strength of the ribosome binding site, iii) the type of RNA polymerase, T5 for the E. coli BL21 host or T7 phage for the BL21 (DE3) strain and iv) the concentration of the IPTG inducer were evaluated.

FIG. 3 shows that for eight expression systems with different plasmid copy numbers (<20 and ˜500 copies), ribosome binding strength (medium binding following mRNA sequence modification and strong binding) and signal peptide SEQ ID NO: 2 (OmpC) and SEQ ID NO: 9 (OmpA), the OD₆₀₀ of cultures induced with IPTG for 4 hours are much lower than those not induced or self-induced with lactose. The optical density at 600 nm of the culture medium in the flask, OD₆₀₀, is measured by spectrophotometry using a standard protocol well known to a person skilled in the art for measuring the optical density and taking into account a blank measurement corresponding to the optical density of the culture medium without bacteria. The inventors conclude that SEQ ID NO: 12 produced too quickly and too extensively is toxic and/or that synthesis thereof represents a significant metabolic load for the bacteria, thus slowing bacterial growth. This result demonstrates the importance of separating the phases of biomass production and expression of SEQ ID NO: 12 and of inducing expression in a sufficiently slow and controlled manner, in order to avoid toxicity, metabolic overload and/or saturation of secretion pathways. Moreover, under these conditions, in particular the high induction with IPTG of the expression of SEQ ID NO: 12, cell toxicity is a lot lower when the signal peptide is used OmpC, compared to the signal peptide OmpA.

Based on the comparative results exemplified above and obtained in cultures carried out in microplates, two vectors associated with the E. coli BL21 (DE3) strain were selected for the final selection carried out in a 5 L bioreactor. The bioreactor is representative of the conditions used for industrial-scale production: BL21 (DE3)/pD431-MR-OmpC-CRM197 (also called BL21 (DE3)/pLM7-OC-CRM) which has a nucleotide sequence SEQ ID NO: 10 and BL21 (DE3)/pD451—SR-OmpC-CRM197 (also called BL21 (DE3)/pHS7-OC-CRM) which has a nucleotide sequence SEQ ID NO: 11. The first expression system comprises the components ensuring a slow expression of SEQ ID NO: 12 (low plasmid copy number, medium ribosome binding), avoiding any toxicity, metabolic overload and/or saturation of secretion pathways while the second comprises the components supporting rapid expression of the protein (high plasmid copy number, strong ribosome binding). However, the second vector was selected in anticipation of low plasmid stability.

The expression levels of both expression systems (BL21 (DE3)/pLM7-OC-CRM and BL21 (DE3)/pHS7-OC-CRM) were compared under conditions representative of industrial conditions. The cultures were grown in 5 L fermenters which are designed, used and controlled in the same way as fermenters for industrial production, especially in terms of cascade, stirring and ventilation. The cultures were grown to high cell densities using “fed-batch” feeding strategies. The expression levels and plasmid stabilities recorded for the various constructs and conditions tested are summarised in Table 2. A condition that combines the highest expression of SEQ ID NO: 12 with a high plasmid stability (condition F05) involves a low plasmid copy number, medium ribosome binding and a temperature on induction of 23° C., which corresponds to a set of factors enabling a slow and controlled expression of SEQ ID NO: 12. Conditions leading to the lowest expression (condition F03) or to the lowest plasmid stability (condition F06) involve a high plasmid copy number and strong ribosome binding, a factor leading to rapid expression of SEQ ID NO: 12 and responsible for toxicity, metabolic overload and/or saturation of secretion pathways.

TABLE 2
expression level and plasmid stabilities in a 5 L reactor - E. coli BL21 (DE3) strain

| | Expression-System [symbol] | | | | Fermentation-conditions [symbol] | | | | | |
|---|---|---|---|---|---|---|---|---|---|---|
| | | Plasmid | Ribosome | | | Temperature | | Growth- | Expression | Plasmid- |
| Fermentation | E. coli | copy- | binding | Signal | | for-induction [symbol] | | rate | level [symbol] | stability |
| batch [symbol] | strain [symbol] | number [symbol] | strength [symbol] | peptide [symbol] | Kanamycin [symbol] | (° C.) [symbol] | pH [symbol] | (h − 1) [symbol] | (g/L) [symbol] | % [symbol] |
| F01 [symbol] | BL21-(DE3 [symbol] | Low [symbol] | Medium [symbol] | Seq ID | Yes [symbol] | 23 [symbol] | 7 [symbol] | 0.1 [symbol] | 2.5 [symbol] | 97% [symbol] |
| F02 [symbol] | | Low [symbol] | Medium [symbol] | No: 2 [symbol] | Yes [symbol] | 28 [symbol] | 7 [symbol] | 0.1 [symbol] | 1 [symbol] | 100% [symbol] |
| F03 [symbol] | | High [symbol] | High [symbol] | | Yes [symbol] | 23 [symbol] | 7 [symbol] | 0.1 [symbol] | 0.4 [symbol] | 7% [symbol] |
| F04 [symbol] | | High [symbol] | High [symbol] | | Yes [symbol] | 28 [symbol] | 7 [symbol] | 0.1 [symbol] | 0.8 [symbol] | 9% [symbol] |
| F05 [symbol] | | Low [symbol] | Medium [symbol] | | No [symbol] | 23 [symbol] | 7 [symbol] | 0.1 [symbol] | 3.1 [symbol] | 94% [symbol] |
| F06 [symbol] | | High [symbol] | High [symbol] | | Yes [symbol] | 23 [symbol] | 7 [symbol] | 0.075 [symbol] | 1.9 [symbol] | 4% [symbol] |
| F07 [symbol] | | High [symbol] | High [symbol] | | No [symbol] | 23 [symbol] | 7 [symbol] | 0.075 [symbol] | 2.1 [symbol] | 7% [symbol] |
| F08 [symbol] | | Low [symbol] | Medium [symbol] | | Yes [symbol] | 23 [symbol] | 7.5 [symbol] | 0.1 [symbol] | 1.4 [symbol] | 98% [symbol] |

[symbol]

indicates data missing or illegible when filed

Based on the comparative results exemplified above, the vector pLM7-OC-CRM was selected for the production of SEQ ID NO: 12 because of its sufficiently high expression level and its high plasmid stability.

### Example 2.—Preparation of the bacterial strain containing the expression vector

Three bacterial strains were transformed with different vectors containing SEQ ID NO: 12 fused to the signal peptide SEQ ID NO: 2 (OmpC):
E. coli BL21, a strain characterised by the absence of the ADE3 prophage and by the use of the T5 bacterial polymerase, the transcription efficiency of which does not match that of the T7 polymerase of the prophage;
E. coli BL21 (DE3), involving induction with IPTG based on the T7 RNA polymerase of the ADE3 prophage;
E. coli BL21 (ADE3) or E. coli BL21 T7XB, a proprietary strain characterised by the deletion of the prophage sequence, with the exception of the genes required for expressing T7 polymerase.

Table 3 compares the different expression systems involving the three strains tested (E. coli BL21, E. coli BL21 (DE3) and E. coli BL21 T7XB) in terms of plasmid copy number, promoter type (T5 or T7) and ribosome binding strength (medium and high), as well as the results obtained during microplate cultures (OD₆₀₀ and cytoplasmic expression level of SEQ ID NO: 12). For E. coli BL21 bacteria, characterised by a lower efficiency of the T5 bacterial polymerase, bacterial growth (OD₆₀₀) is not significantly (or only slightly) affected during IPTG induction, while expression levels evaluated by immunoblot analysis are low, with the exception of that obtained for the strain transformed by a vector involving a high copy number and strong ribosome binding. No toxicity on bacterial growth and significant expression are observed during IPTG induction for the E. coli BL21 (DE3) strain transformed by a plasmid involving a low copy number and medium ribosome binding; all the other constructs being subject to toxicity and/or low expression. The results obtained for the E. coli BL21 T7XB strain are similar to those obtained for the E. coli BL21 (DE3) strain.

TABLE 3
OD₆₀₀ and periplasmic expression levels of cells inoculated by
the different expression systems after induction with IPTG.

| E. coli | | | Origin of | Strength | | Periplasmic |
|---|---|---|---|---|---|---|
| strain | Promoter | Plasmid | replication | of RBS | OD₆₀₀ Results | expression results |
| BL21 | T5 | pD421-MR | P15a (10-12 | Medium | High OD₆₀₀ (>15) | No expression |
| BL21 | T5 | pD421-SR | copies/cell) | High | High OD₆₀₀ (>15) | Low expression |
| BL21 | T5 | pD441-MR | pUC (>500 | Medium | High OD₆₀₀ (>15) | Low expression |
| BL21 | T5 | pD441-SR | copies/cell) | High | Medium OD₆₀₀ (5-10) | Expression detected |
| BL21 (DE3) | T7 | pD431-MR | P15a (10-12 | Medium | Medium OD₆₀₀ (5-10) | Expression detected |
| BL21 (DE3) | T7 | pD431-SR | copies/cell) | High | Low OD₆₀₀- Toxicity | Low expression |
| BL21 (DE3) | T7 | pD451-MR | pUC (>500 | Medium | Low OD₆₀₀- Toxicity | Low expression |
| BL21 (DE3) | T7 | pD451-SR | copies/cell) | High | Low OD₆₀₀- Toxicity | Low expression |
| BL21 (T7XB) | T7 | pD431-MR | P15a (10-12 | Medium | Medium OD₆₀₀(5-10) | Expression detected |
| BL21 (T7XB) | T7 | pD431-SR | copies/cell) | High | Low OD₆₀₀- Toxicity | Low expression |

The expression levels as well as plasmid stabilities were determined for the three bacterial strains (E. coli BL21, E. coli BL21 (DE3) and E. coli BL21 T7XB) transformed with different vectors containing SEQ ID NO: 12 fused to the signal peptide SEQ ID NO: 2 under conditions representative of industrial conditions. The cultures were grown in 5 L fermenters which are designed, used and controlled in the same way as fermenters for industrial production, especially in terms of cascade, stirring and ventilation. The cultures reach high cell densities using “fed-batch” feeding strategies. The expression levels, the plasmid stabilities recorded for the different constructs and the conditions tested are summarised in Table 4. The four constructs involving the E. coli BL21strain characterised by the lower efficiency of the T5 bacterial polymerase have high expression levels between 1.02 and 2.46 g/L and high plasmid stability (between 97 and 100%), regardless of the plasmid copy number and the ribosome binding strength. For the E. coli BL21 (DE3) strain, only constructs involving a low plasmid copy number and medium ribosome binding have high expression levels between 1.00 and 3.10 g/L and high plasmid stability (between 94 and 100%). For the E. coli BL21 (T7XB) strain characterised by the deletion of the prophage sequence, with the exception of the genes required for expressing T7 polymerase, a high expression level (1.62 g/L) and a high plasmid stability (97%) are recorded for the construct involving a low plasmid copy number and medium ribosome binding, whereas a low expression level (0.62 g/L) and a low plasmid stability (23%) are observed with the strain transformed by a vector involving a low plasmid copy number and strong ribosome binding.

TABLE 4
expression levels and plasmid stabilities in a 5 L reactor: E. coli BL21, E. coli BL21 (DE3) and E. coli BL21 T7XB strains

| | Expression-System [symbol] | | | | Fermentation-conditions [symbol] | | | | | |
|---|---|---|---|---|---|---|---|---|---|---|
| | | Plasmid- | Ribosome- | | | Temperature | | Growth- | Expression | Plasmid- |
| Fermentation- | E. coli- | copy- | binding- | Signal | | for-induction [symbol] | | rate | level [symbol] | stability |
| batch [symbol] | strain [symbol] | number [symbol] | strength [symbol] | peptide [symbol] | Kanamycin [symbol] | (° C.) [symbol] | pH [symbol] | (h − 1) [symbol] | (g/L) [symbol] | % [symbol] |
| F09 [symbol] | BL21 [symbol] | Low [symbol] | Medium [symbol] | Seq ID | No [symbol] | 23 [symbol] | 7 [symbol] | 0.075 [symbol] | 1.02 [symbol] | 100% [symbol] |
| F10 [symbol] | | Low [symbol] | High [symbol] | No: 2 [symbol] | No [symbol] | 23 [symbol] | 7 [symbol] | 0.075 [symbol] | 1.78 [symbol] | 100% [symbol] |
| F11 [symbol] | | High [symbol] | Medium [symbol] | | No [symbol] | 23 [symbol] | 7 [symbol] | 0.075 [symbol] | 2.46 [symbol] | 97% [symbol] |
| F12 [symbol] | | High [symbol] | High [symbol] | | No [symbol] | 23 [symbol] | 7 [symbol] | 0.075 [symbol] | 1.26 [symbol] | 97% [symbol] |
| F01 [symbol] | BL21 (DE3) [symbol] | Low [symbol] | Medium [symbol] | | Yes [symbol] | 23 [symbol] | 7 [symbol] | 0.1 [symbol] | 2.50 [symbol] | 97% [symbol] |
| F02 [symbol] | | Low [symbol] | Medium [symbol] | | Yes [symbol] | 28 [symbol] | 7 [symbol] | 0.1 [symbol] | 1.00 [symbol] | 100% [symbol] |
| F03 [symbol] | | High [symbol] | High [symbol] | | Yes [symbol] | 23 [symbol] | 7 [symbol] | 0.1 [symbol] | 0.40 [symbol] | 7% [symbol] |
| F04 [symbol] | | High [symbol] | High [symbol] | | Yes [symbol] | 28 [symbol] | 7 [symbol] | 0.1 [symbol] | 0. [symbol] 0 [symbol] | 9% [symbol] |
| F05 [symbol] | | Low [symbol] | Medium [symbol] | | No [symbol] | 23 [symbol] | 7 [symbol] | 0.1 [symbol] | 3.10 [symbol] | 94% [symbol] |
| F06 [symbol] | | High [symbol] | High [symbol] | | Yes [symbol] | 23 [symbol] | 7 [symbol] | 0.075 [symbol] | 1.90 [symbol] | 4% [symbol] |
| F07 [symbol] | | High [symbol] | High [symbol] | | No [symbol] | 23 [symbol] | 7 [symbol] | 0.075 [symbol] | 2.10 [symbol] | 7% [symbol] |
| F08 [symbol] | | Low [symbol] | Medium [symbol] | | Yes [symbol] | 23 [symbol] | 7.5 [symbol] | 0.1 [symbol] | 1.40 [symbol] | 98% [symbol] |
| F15 [symbol] | | Low [symbol] | Medium [symbol] | | No [symbol] | 23 [symbol] | 6.5 [symbol] | 0.1 [symbol] | 0.56 [symbol] | 89% [symbol] |
| F16 [symbol] | | Low [symbol] | High [symbol] | | No [symbol] | 23 [symbol] | 7 [symbol] | 0.1-0.075 [symbol] | 1.42 [symbol] | 76% [symbol] |
| F13 [symbol] | T7 (XB) [symbol] | Low [symbol] | Medium [symbol] | | No [symbol] | 23 [symbol] | 7 [symbol] | 0.1 [symbol] | 1.62 [symbol] 2 [symbol] | 97% [symbol] |
| F14 [symbol] | | Low [symbol] | High [symbol] | | No [symbol] | 23 [symbol] | 7 [symbol] | 0.1-0.075 [symbol] | 0. [symbol] 2 [symbol] | 23% [symbol] |

[symbol]

indicates data missing or illegible when filed

However, the third E. coli BL21 T7XB strain, characterised by the deletion of the prophage sequence, with the exception of the genes required for expressing T7 polymerase, was selected for the production of SEQ ID NO: 12. The inventors determined that controlling expression using an expression system based on T5 bacterial RNA polymerase (E. coli BL21), sometimes leads to leakage and reduced robustness, and, secondly, that the presence of the 2DE3 prophage in the E. coli BL21 (DE3) strain presents a potential reactivation of the lytic cycle and therefore a significant industrial risk.

### Example 3.—Periplasmic expression of CRM197 in E. coli

The vector pLM7-OC-CRM combined with the E. coli BL21 T7XB strain provides optimal periplasmic production yields of SEQ ID NO: 12. Optimal periplasmic production of CRM197 depends on the right balance between the expression level of the protein CRM197, the nature of the signal peptide and the plasmic stability of the vector. An expression level that is too high may cause high cytoplasmic production of the protein CRM197 in insoluble form, decreasing cell viability, without causing significant periplasmic production.

The steps below describe, in one exemplary embodiment of the present invention, the various conditions used to produce SEQ ID NO: 12 during culture in a fermenter inoculated with the E. coli BL21 T7XB strain transformed by the plasmid pLM7-OC-CRM:
Strain used: E. coli BL21 T7XB/pLM7-OC-CRM (SEQ ID NO: 10).
Step 1: preculture in a flask

Volume of the flask: 500 ml to 2 L, Inoculation OD₆₀₀: 0.005, culture medium: YGP, kanamycin: 25 mg/L, temperature: 37° C., stirring: 270 rpm, culture duration: 7 hours+/−1 hour.
Step 2: culture in “batch” mode

Volume of the bioreactor: 50 L, culture medium: GYPSCI containing 10 g/L glycerol and 5 g/L yeast extract, temperature: 37° C., pH: 7.0, feeding with GYPSCI medium containing 400 g/L glycerol and 217.5 g/L yeast extract started at time t=0 hours, with a linear increase in the feed rate from 0 to 127 g/h between the time t=0 and t=9 hours 30 minutes.
Step 3: culture in “fed-batch” mode

Feeding with GYPSCI medium started at time t=9 hours 30 minutes, with an exponential growth rate of 0.15 h−1, temperature: 37° C., pH: 7.0, exponential feed stopped after +/−10 hours when OD₆₀₀=60+/−2.
Step 4: induction phase in “fed-batch” mode

An exponential growth rate of 0.1 hours-1 for 6 hours was used, then a constant feed rate for 14 hours, induction started by adding IPTG so as to obtain a concentration in the reactor of 2 mM, temperature: 23° C., pH: 7.0, total time of the induction phase: 20 hours.
Step 5: Collection and centrifugation step

Step 4 (induction phase) stopped after 20 hours, temperature decreased to 15° C., contents of the bioreactor transferred into centrifuge containers, centrifugation at 7000 rpm (9000-9500 g), cell pellets collected, transferred into plastic bags and said plastic bags stored at −20° C.

The impact of changing the various culture parameters (presence of kanamycin in the culture medium, temperature and pH on induction) was studied and the results shown in Table 5. Under the conditions optimised by the inventors, the absence of kanamycin did not significantly affect the expression level of SEQ ID NO: 12 or plasmid stability (comparison of conditions F01, F05 and F13). The temperature applied on induction is a parameter with little impact on expression level but no impact on plasmid stability (comparison of conditions F01 and F02). The pH is a parameter with significant impact on expression level and, to a lesser extent, plasmid stability; a pH of 6.5 is less favourable, whereas a pH of 7.0 is optimal for both criteria. The absence of kanamycin, a temperature decrease from 28 to 23° C. on induction and a pH of 7.0 kept constant throughout the culture steps enable a good production yield of SEQ ID NO: 12.

TABLE 5
expression level and plasmic stabilities in a 5 L reactor: impact of kanamycin, temperature and pH

| | Expression System [symbol] | | | | Fermentation conditions [symbol] | | | | | |
|---|---|---|---|---|---|---|---|---|---|---|
| | | Plasmid- | Ribosome- | | | Temperature | | Growth- | Expression | Plasmid- |
| Fermentation- | E.-coli- | copy | binding- | Signal- | | for-induction [symbol] | | rate | level [symbol] | stability |
| batch [symbol] | strain [symbol] | number [symbol] | strength [symbol] | peptide [symbol] | Kanamycin [symbol] | (° C.) [symbol] | pH [symbol] | (h − 1) [symbol] | (g/L) [symbol] | % [symbol] |
| F01 [symbol] | BL21 (DE3) [symbol] | Low [symbol] | Medium [symbol] | Seq ID | Yes [symbol] | 23 [symbol] | 7 [symbol] | 0.1 [symbol] | 2.50 [symbol] | 97% [symbol] |
| F02 [symbol] | BL21 (DE3) [symbol] | | | No: 2 [symbol] | Yes [symbol] | 28 [symbol] | 7 [symbol] | | 1.00 [symbol] | 100% [symbol] |
| F05 [symbol] | BL21 (DE3) [symbol] | | | | No [symbol] | 23 [symbol] | 7 [symbol] | | 3.10 [symbol] | 94% [symbol] |
| F08 [symbol] | BL21 (DE3) [symbol] | | | | Yes [symbol] | 23 [symbol] | 7.5 [symbol] | | 1.40 [symbol] | 98% [symbol] |
| F15 [symbol] | BL21 (DE3) [symbol] | | | | No [symbol] | 23 [symbol] | 6.5 [symbol] | | 0.55 [symbol] | 89% [symbol] |
| F13 [symbol] | T7 (X8) [symbol] | | | | No [symbol] | 23 [symbol] | 7 [symbol] | | 1.62 [symbol] | 97% [symbol] |

[symbol]

indicates data missing or illegible when filed

### Example 4.—Periplasmic Extraction of SEQ ID NO: 12

After the various culture and induction steps, the E. coli BL21 T7XB bacteria transformed by the vector pLM7-OC-CRM (SEQ ID NO: 10) are centrifuged and the cellular slurries are frozen at −20° C. Periplasmic extraction begins by thawing the cellular slurries of 1350 g for 15 hours+/−5 hours at 6° C.±/−2° C. The cellular slurries are diluted at a 2:1 (v/w) ratio in a hypertonic solution (50 mM Tris, 5 mM EDTA, 20% Sucrose, pH 8.0), mixed for 15 minutes and pumped using a first pump into a mixer through a diffusion ring, the flow rate of which is 121 ml/min.

An osmotic shock of the cell suspension is then carried out by diluting the cell suspension contained in the mixer with a hypotonic solution (5 mM MgCl₂) which is pumped with a second pump into the mixer containing the cell suspension with a flow rate of 279 ml/min and a dilution ratio of 2.3:1 (v/v) of hypotonic solution. The temperature of the hypotonic solution is between 4 and 8° C. The volume of the cell suspension in the mixer is 200 ml and the residence time of the cell suspension in the mixer is 30 seconds. A third pump extracts the cell suspension from the mixer, which is collected in centrifuge containers, at a flow rate of 400 ml/min, so as to maintain a constant cell suspension volume of 200 ml in the mixer. A 1350 g weight of cellular slurries yields approximately 13.4 litres of osmotically shocked cell suspension. The osmotically shocked cell suspension is centrifuged at 8000 rpm for 20 minutes (g=9379 and K factor=3242). The pellets are removed and the supernatant is clarified by filtration.

### Example 5.—Clarification of the Supernatant Containing the Components of the Periplasmic Space and SEQ ID NO: 12

Clarifying the supernatant resulting from the centrifugation of the cell suspension after osmotic shock (see Example 4) consists of two successive filtration steps. For the first filtration, the supernatant is pumped at a rate of 400 ml/min through a filter with a mesh size of 0.8-0.45 μm and an area of 0.2 m². The once-filtered solution is then pumped at a rate of 400 ml/min through a second filter with a mesh size of 0.45-0.22 μm and an area of 0.1 m², providing a solution containing a clarified periplasmic extract.

It is to be understood that the present invention is in no way limited to the embodiments described above and that modifications may be made without departing from the scope of the accompanying claims.

1. Expression vector in a Gram-negative prokaryote with the following features:
(i) a low copy number origin of replication, said low copy number being defined by a plasmid copy number of 20 per cell or less,
(ii) a promoter sequence inducible with an exogenous inducing agent,
(iii) a medium strength ribosome binding site, said medium strength ribosome binding site corresponds to an mRNA sequence modified to have a reduced ribosome binding,
(iv) a sequence encoding a protein having at least 95% identity to the full-length SEQ ID NO: 12.
(v) a sequence encoding a signal peptide having a sequence identity to SEQ ID NO: 2 of at least 95%, said signal peptide being arranged to enable the targeting of said SEQ ID NO: 12 towards a periplasmic space.

2. Expression vector according to claim 1, wherein the glutamic acid residue at position 52 of SEQ ID NO: 12 is retained.

3. Expression vector according to claim 1 or claim 2, wherein the inducible promoter sequence has at least 95% identity to SEQ ID NO: 5 over the entire length and is specifically recognised by a T7 phage RNA polymerase.

4. Expression vector according to any one of claims 1 to 3, wherein said medium strength ribosome binding site is defined by a consensus sequence SEQ ID NO: 6 or by the consensus sequence SEQ ID NO: 6 comprising an addition and/or a deletion and/or a point mutation of 1 or 2 or 3 or 4 or 5 nucleotides, said SEQ ID NO: 6 being located upstream of a translation initiation site.

5. Transformed strain of E. coli bacteria comprising the expression vector according to any one of the preceding claims.

6. Transformed strain of bacteria according to claim 5, being the E. coli BL21 strain defined by a deletion of the ADE3 prophage with the exception of the genes required for expressing T7 phage RNA polymerase.

7. Method for producing in the periplasmic space a peptide having at least 95% identity to SEQ ID NO: 12 over the entire length thereof comprising:
(i) transforming a strain of E. coli by a vector enabling expression of said SEQ ID NO: 12 in the periplasmic space of said E. coli, said expression being inducible by an exogenous agent,
(ii) culturing the transformed E. coli strain at a substantially constant temperature, for example 37° C.±1° C., and a constant pH under conditions where SEQ ID NO: 12 is not synthesised by said transformed E. coli strain,
(iii) an induction phase for a predetermined time period in the presence of said exogenous agent and at a reduced temperature to synthesise and secrete SEQ ID NO: 12 in the periplasmic space in a slow and controlled manner,
(iv) optionally harvesting the culture after said induction phase and after cooling,
(v) optionally collecting the cultured bacteria containing SEQ ID NO: 12 in the periplasmic space,
(vi) optionally performing periplasmic extraction of said SEQ ID NO: 12 including separating a solid pellet containing the bacteria and a supernatant containing the components of the periplasmic space and SEQ ID NO: 12, and
(vii) optionally purifying said protein SEQ ID NO: 12.

8. Method according to claim 7, wherein the E. coli strain is the BL21 strain defined by a deletion of the ADE3 prophage with the exception of the genes required for expressing T7 phage RNA polymerase.

9. Method according to claim 7 or claim 8 wherein the production in the periplasmic space of a peptide having at least 95% identity to SEQ ID NO: 12 over the entire length thereof is provided by a targeting signal peptide in the periplasmic space, said signal peptide having a sequence identity to SEQ ID NO: 2 of at least 95%.

10. Method according to any one of claims 7 to 9, wherein the reduced temperature is 23° C.±0.5° C. and/or the predetermined time period of the induction phase is between 15 and 25 hours, preferably between 18 and 22 hours and more preferably around 20 hours and/or the pH of the induction phase is between 6.8 and 7.5, preferably between 7.0 and 7.2.

11. Method according to any one of claims 7 to 10, wherein expression inducible by an exogenous agent is performed by an on/off induction system and wherein said exogenous agent is isopropyl-β-D-1-thiogalactopyranoside.

12. Method according to any one of claims 7 to 11, wherein the culture and the induction phase are antibiotic-free.

13. Method according to any one of claims 7 to 12, wherein the expression vector is of a low copy number defined by a plasmid copy number of 20 per cell or less and/or the ribosome binding sequence is of medium strength.

14. Method according to any one of claims 7 to 13, wherein the purification of the protein SEQ ID NO: 12 comprises the successive steps of:
(i) clarifying the supernatant from the periplasmic extraction containing the components of the periplasmic space and the protein by at least one filtration step, preferably two filtration steps and recovering a filtrate comprising SEQ ID NO: 12, optionally,
(ii) a series of chromatographic steps arranged to recover SEQ ID NO: 12 in a fraction to be retained and the contaminants in one or more fractions to be removed,
(iii) optionally, removing residual endotoxins, and
(iv) optionally, sterilising the endotoxin-free fraction comprising SEQ ID NO: 12 to be retained.

15. Method for selecting a signal peptide so as to optimise production of a protein of interest in the periplasmic space of E. coli bacteria, wherein:
a genetic construct is generated, comprising a rhamnose-inducible promoter having at least 95% sequence identity to SEQ ID NO: 7 controlling the expression of said protein of interest arranged with said signal peptide to be tested,
said E. coli is transformed with said genetic construct,
several parallel cultures of said transformed E. coli are produced in a rhamnose-free culture medium,
said transformed E. coli is cultured in the presence of a determined concentration of rhamnose so as to obtain a low induction of the expression of said protein of interest and another culture of said transformed E. coli in the presence of another determined concentration of rhamnose so as to obtain a high induction of the expression of said protein of interest and,
the abundance of the protein of interest in the periplasmic space is compared and, optionally, the deleterious effects to metabolism selected from the group consisting of plasmid loss, reduction in bacterial biomass, formation of cytosolic aggregates or degradation products and partial cleavage of the signal peptide, are quantified and,
the signal peptide is selected according to the abundance of the protein of interest in the periplasmic space and, optionally, by rejecting signal peptides associated with excessively high deleterious effects.
